(19) United States
(12) Patent Application Publication
(10) Pub. No.: US 2008/0083046 A1
(43) Pub. Date: Apr. 10, 2008

(54) Apparel advertising sun visor

(76) Inventor: Gordon Kent Matthias, Conroe, TX (US)

Correspondence Address: Gordon Kent Matthias, 545 F.M. 1488 #2206, Conroe, TX, 77384, US

(21) Appl. No.: 11/906,956

(22) Filed: Oct. 4, 2007

Related U.S. Application Data
(60) Provisional application No. 60/849,180, filed on Oct. 4, 2006.

(51) Int. Cl. A61F 9/00; A41D 13/00

(52) U.S. Cl. 2/12; 2/455

(57) ABSTRACT

An improved wearing apparel type sun visor made of card stock paper such as is found in but not limited to twelve pack cartons of soft drink beverages and made in one piece and which attaches to most eye glasses by way of two holes on each side through which said eye glasses' ear pieces are threaded for support and which is intended to display printed advertisements.

### CROSS REFERENCE TO RELATED APPLICATIONS

Applicant claims the benefit of his provisional patent application filed Oct. 4, 2006, Appl. No. 60/849,180 titled “The Ad-Vizr.

### DESCRIPTION OF INVENTION

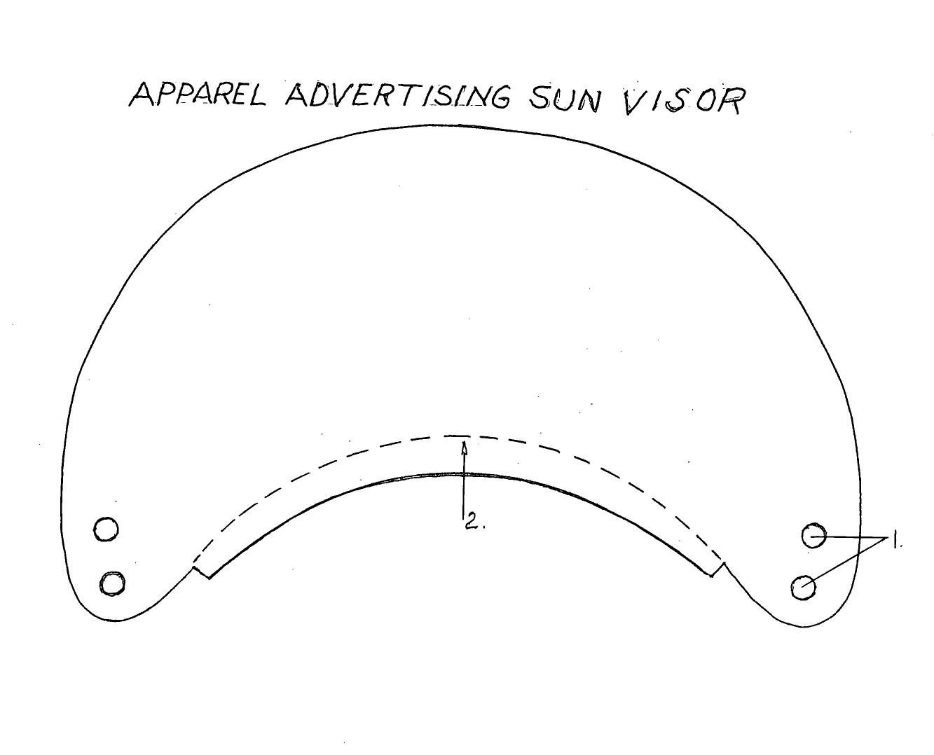

A wearing apparel type sun visor which adapts to either prescription eye glasses, or prescription or non-prescription sun glasses by two holes (FIG. 1) on each side of the visor through which the ear support parts of the glasses are threaded and which are placed at angles to each other so as to give the visor proper stability, support and attitude thus providing shade to the wearer's eyes and face. The visor contains a scored or perforated arched line (FIG. 2) which when folded downward causes the visor to arch upward at the middle thereby creating more upward peripheral vision for the wearer as well as a bumper guard for the wearer's forehead and more stability of the visor itself. The visor and all its relative aspects including scoring and/or perforating are created in a cutting or punch press of a kind normally associated with the cutting and scoring of product cartons.

The visor is made of paper, plastic or some other flexible material and can be adorned with advertisements or otherwise artistic decoration and can be made as separates or incorporated, by scoring or perforating, into other products of like material such as soft drink or other product cartons as a “pop-out”.

### BACKGROUND

The visor being claimed herein is unique to other apparel type visors in that it is of one part only and can be manufactured with one die cut on card gauge paper as is found in the average carton of a given product where the carton has a panel of sufficient size to accommodate the visor's predetermined dimensions. This visor attaches to eye glasses but, unlike other visors found, no clips or other parts are needed. The glasses ear pieces are simply threaded through the holes which are also created in the die cut.

This visor is also different in the fact that in addition to a sun visor's function of shading the wearer's eyes and face from any overhead light source, this visor will display an advertisement of a given product and can be distributed to its intended wearer as a separate; ie. a hand out at a sporting or other public event or incorporated into a product carton such as a twelve pack of a given type of beverage or any other product where a carton is used in packaging for product sale or give away.

In addition, and because of its simplicity and type of material used along with method of manufacturing, my visor will cost, on a comparative per unit per run basis, a fraction of similar costs associated with other apparel type sun visors found.

1. I, hereby, claim an apparel type visor; titled: “Apparel Advertising Sun Visor” to be an original invention which I alone have created and to which I am entitled a patent to be issued by the U.S. Patent and Trademark Office pursuant to U.S. patent laws.
